(19) United States
(12) Patent Application Publication
(10) Pub. No.: US 2009/0168016 A1
(43) Pub. Date: Jul. 2, 2009

(54) VISION RECOVERY TRAINING DEVICE

(75) Inventors: Eiichi Ohkawa, Kanagawa (JP); Hisashi Hagiwara, Kanagawa (JP); Fumiaki Emoto, Kanagawa (JP); Saho Yamada, Kanagawa (JP)

Correspondence Address: PEARNE & GORDON LLP, 1801 EAST 9TH STREET, SUITE 1200, CLEVELAND, OH, 44114-3108, US

(73) Assignees: MATSUSHITA ELECTRIC INDUSTRIAL CO., LTD., Osaka (JP); TECHNOMASTER CO., LTD., KANAGAWA (JP)

(21) Appl. No.: 11/571,302

(22) PCT Filed: Jul. 11, 2005
(86) PCT No.: PCT/JP2005/012767

§ 371 Date: Dec. 27, 2006

(30) Foreign Application Priority Data
Jul. 14, 2004 (JP) 2004-207076

(51) Int. Cl. A61B 3/00

(52) U.S. Cl. 351/203

(57) ABSTRACT

An object of the present invention is to provide a visual training apparatus in which a trainee can make accommodation of the trainee's eye easily when the trainee is training. The visual training apparatus includes a display member 1, an optical system 2, a base 3 and a housing 4. The display member 1 displays a training image, and is fixed to the base 3 so as to cross an optic axis 51 of a trainee 5 substantially perpendicularly thereto. The optical system 2 includes a lens 21, a feed screw 22 and a motor 23. When the motor 23 is driven, the lens 21 moves along the optic axis 51 with the aid of the feed screw 22. Thus, the optical characteristic of the optical system 2 is changed so that the optical distance between the eye position of the trainee 5 and the display member 1 can be changed. The display member 1 displays a training image having dimensions proportional to the optical distance between the eye position of the trainee and the display member 1.

### TECHNICAL FIELD

The present invention relates to a visual training apparatus for training an accommodation function of an eye.

### BACKGROUND ART

It can be regarded, as one of the causes of low vision, that the accommodation function of an eye decreases due to tonic accommodation or weakness of accommodation muscle (ciliary muscle) of the eye. When exercises are given to this muscle, the vision can be expected to recover. This method is to give exercises to the ciliary muscle to thereby activate the ciliary muscle and hence activate the accommodation function for the recovery of the vision.

Patent Document 1 discloses a vision recovery apparatus for activating the operation of this accommodation muscle to thereby improve the effect on the recovery of the vision. In this vision recovery apparatus, a regard plate on which a mark is indicated is reciprocated to make the mark close to and away from a user. When the user of this apparatus gives regard on this mark, the eye focus moves continuously between far and near. Accordingly the accommodation muscle is trained efficiently.

In this vision recovery apparatus, the regard plate where the mark is indicated is moved to change the distance between the user and the mark. Accordingly, the mechanism is complicated and extensive. In addition, due to a movable portion provided in the apparatus, the apparatus lacks durability and maintainability.

Further, the mark indicated in the regard plate has a fixed size. Accordingly, there is a case where the user hardly focuses on the mark. That is, when the distance between the user and the mark is short, the area where an image of the mark is formed on the retina of the user is so large that the amount of information input to the brain increases to prevent the user from easily making accommodation of the eye.

On the other hand, there has been proposed a visual training apparatus in which the distance between a trainee and an indicator for the trainee to give regard on is changed by use of an optical system so that the apparatus can be miniaturized (see Patent Document 2).

In the visual training apparatus disclosed in Patent Document 2, the characteristic of the optical system disposed between the trainee and a display panel where the indicator is displayed is changed to change the optical distance between the eye position of the trainee and the indicator. In this apparatus, in order to emphasize perspective given to the trainee, the indicator can be displayed to be larger when the optical distance is shorter, and the indicator can be displayed to be smaller when the optical distance is longer.

However, when the perspective of the indicator is emphasized, a shortsighted trainee will have no feeling of wrongness even if the trainee does not focus on the indicator displayed away from the trainee, and a longsighted trainee will have no feeling of wrongness even if the trainee does not focus on the indicator displayed closely to the trainee. As a result, trainees cease to focus on the indicator so that the effect of training cannot be expected. When the size of the indicator is fixed, there arises a problem similar to that of the apparatus disclosed in Patent Document 1. That is, when the distance between the user and the indicator is short, the area where an image of the indicator is formed on the retina of the user is so large that the amount of information input to the brain increases to prevent the user from easily making accommodation of the eye.

### Patent Document 1: JP-A-6-339501

### Patent Document 2: JP-A-8-257077

### DISCLOSURE OF THE INVENTION

### Problems that the Invention is to Solve

The present invention was developed in consideration of the aforementioned situation. An object of the invention is to provide a visual training apparatus in which a trainee can make accommodation of the trainee's eye easily when the trainee is training.

### Means for Solving the Problems

A visual training apparatus according to the present invention serves to train an accommodation function of an eye, including:

a display member that is fixedly disposed to cross a visual axis of a trainee substantially perpendicularly thereto;

an optical system that is disposed between an eye position of the trainee and the display member;

a display control portion that controls display of a training image on the plate-like display member; and

an optical characteristic changing portion that changes an optical characteristic of the optical system to change an optical distance between the eye position of the trainee and the display member,

wherein the display control portion displays on the display member the training image having a dimension proportional to the optical distance between the eye position of the trainee and the display member.

According to the present invention, there is no substantial change in the dimensions of the training image occupying the field of view of the trainee. Thus, the accommodation function of the eye can be controlled easily.

The visual training apparatus according to the present invention includes a visual training apparatus in which the display control portion displays on the display member the training images having the dimensions proportional to the optical distance between the eye position of the trainee and the display member and being similar to one another. According to the present invention, there is no substantial change in the dimensions and the shape of the training image watched by the trainee. Thus, the accommodation function of the eye can be controlled easily.

The visual training apparatus according to the present invention includes a visual training apparatus in which the optical system includes a lens, and the optical characteristic changing portion includes a driving portion which moves a position of the lens. According to the present invention, the optical distance between the eye position of the trainee and the display member can be changed largely in spite of a small moving distance of a movable portion. Thus, the apparatus can be made small in size and light in weight.

The visual training apparatus according to the present invention includes a visual training apparatus in which the optical system is a varifocal lens. According to the present invention, the lens system can be driven easily, and further, the apparatus can be made small in size and light in weight.

The visual training apparatus according to the present invention includes a visual training apparatus in which the plate-like display member is a liquid crystal display panel. According to the present invention, the training image can be changed easily and surely, and the apparatus can be made small in size and light in weight.

The visual training apparatus according to the present invention includes a visual training apparatus in which the plate-like display member is an organic EL display panel in which organic EL devices are formed on a substrate. According to the present invention, the organic EL display panel can change the training image easily and surely. In addition, the organic EL display panel emits light by itself. Accordingly, no illumination light is required, but the apparatus can be made small in size and light in weight.

### EFFECT OF THE INVENTION

As is apparent from the aforementioned description, according to the present invention, it is possible to provide a visual training apparatus in which a trainee can make accommodation of the trainee's eye easily when the trainee is training.

### BRIEF DESCRIPTION OF THE DRAWINGS

FIG. 1 A diagram showing the schematic configuration of a visual training apparatus according to an embodiment of the present invention.

FIG. 2 A schematic function block diagram of a control unit for controlling the operation of the visual training apparatus according to the embodiment of the present invention.

FIG. 3 Views showing examples of training images to be displayed by the visual training apparatus according to the embodiment of the present invention.

### DESCRIPTION OF REFERENCE NUMERALS

1 . . . display member
2 . . . optical system
21 . . . lens
22 . . . feed screw
23 . . . motor
3 . . . base
4 . . . housing
41 . . . window portion
5 . . . trainee
51 . . . optic axis
61 . . . main control portion
62 . . . lens movement control portion
63 . . . display control portion
64 . . . operating portion

### BEST MODE FOR CARRYING OUT THE INVENTION

An embodiment of the present invention will be described below with reference to the drawings.

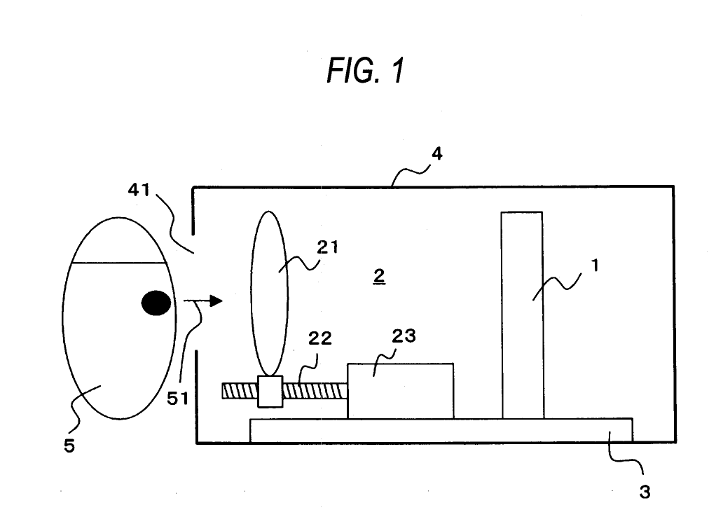

FIG. 1 shows the schematic configuration of a visual training apparatus according to an embodiment of the present invention. The visual training apparatus of FIG. 1 is designed to include a display member 1, an optical system 2, a base 3 and a housing 4.

The display member 1 serves to display a training image. The display member 1 is fixed to the base 3 so as to cross an optic axis 51 of a trainee 5 substantially perpendicularly thereto. The display member 1 is, for example, a liquid crystal display panel having a backlight on the back surface. The display member 1 displays a training image having a predetermined shape and predetermined dimensions in accordance with a control signal from a display control unit which will be described later.

The display member 1 is not limited to the liquid crystal display panel. Various display devices such as a CRT display device, an organic EL display panel in which organic EL devices are formed on a substrate, etc. can be used as the display member 1.

The organic EL display panel emits light by itself. Thus, the organic EL display panel does not require illumination light but can be made small in size and light in weight.

The optical system 2 is disposed between the eye position of the trainee 5 and the display member 1. The optical system 2 includes a lens 21, a feed screw 22 and a motor 23. When the motor 23 is driven, the lens 21 moves along the optic axis 51 with the aid of the feed screw 22. Accordingly, the optical characteristic of the optical system 2 is changed so that the optical distance between the eye position of the trainee 5 and the display member 1 can be changed. The motor 23 is driven based on a control signal from a lens movement control portion which will be described later.

The base 3 is disposed inside the housing 4. The base 3 supports the display member 1 and the optical system 2. The housing 4 covers the whole of the plate like display member 1 except a window portion 41 so as to prevent outside light from entering the housing 4. The trainee 5 can look in through the window portion 41.

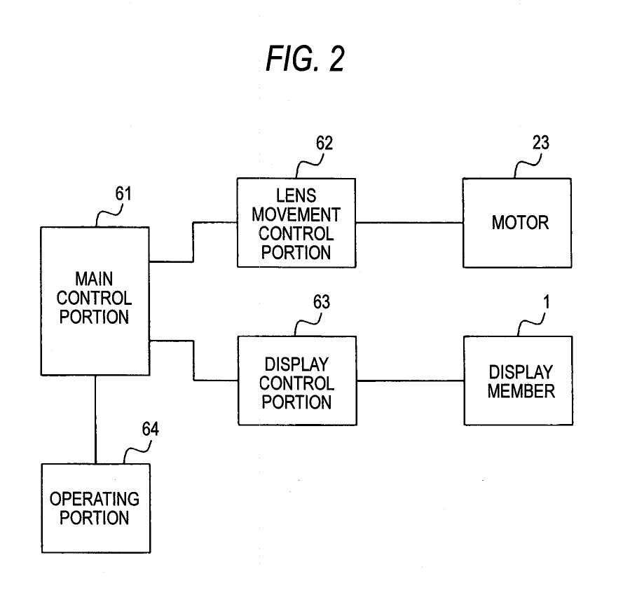

FIG. 2 shows a schematic function block diagram of a control unit for controlling the operation of the visual training apparatus. The control unit has a main control portion 61, a lens movement control portion 62, a display control portion 63 and an operating portion 64.

The main control portion 61 controls the operation of the visual training apparatus as a whole. The main control portion 61 controls the lens movement control portion 62 and the display control portion 63 based on an instruction from the operating portion 64. The lens movement control portion 62 outputs a driving signal to the motor 23 so as to control the movement of the lens 21. In that event, the lens movement control portion 62 acquires information corresponding to settings of the operating portion 64 from the main control portion, and controls the movable range and the moving velocity of the lens 21. The lens 21 may be moved continuously or intermittently. When the lens 21 is moved intermittently, a training image is not moved for a predetermined time.

The display control portion 63 drives the display member 1 so as to make the display member 1 display a training image having a predetermined shape and predetermined dimensions. The display control portion 63 displays a training image having dimensions proportional to the optical distance between the eye position of the trainee and the display member 1. Information about the optical distance between the eye position of the trainee and the display member 1 is acquired from the main control portion 61. Data for displaying the training image having dimensions proportional to the optical distance between the eye position of the trainee and the display member 1 may be calculated every time when the training image is displayed. Alternatively, data stored in advance may be read and sent to the display member 1.

The operating portion 64 is operated by the trainee 5 so as to set the operating mode of the visual training apparatus, and so on. For example, the operating portion 64 sets the movable range and the moving velocity of the lens 21, the kind of training image, and so on.

When visual training is performed using the visual training apparatus shown in FIG. 1, the trainee 5 looks in through the window portion 41 of the housing 4, and gives regard on the training image displayed on the display member 1. The optical distance to the training image changes in accordance with the movement of the lens 21. Accordingly, the trainee 5 watches the training image at a variable optical distance. Thus, the trainee 5 makes efforts to adjust the eye's focus in accordance with the change of the optical distance so as to activate the accommodation function of the eye such as the ciliary muscle. It is therefore possible to expect the accommodation function to be improved.

It is preferable that the optical distance between the eye position of the trainee 5 and the training image displayed in the closest position to the trainee 5 is made as long as the near point distance of a person having a normal vision. It is also preferable that the optical distance between the eye position of the trainee 5 and the training image displayed in the farthest position from the trainee 5 is made as long as the far point distance of a person having a normal vision.

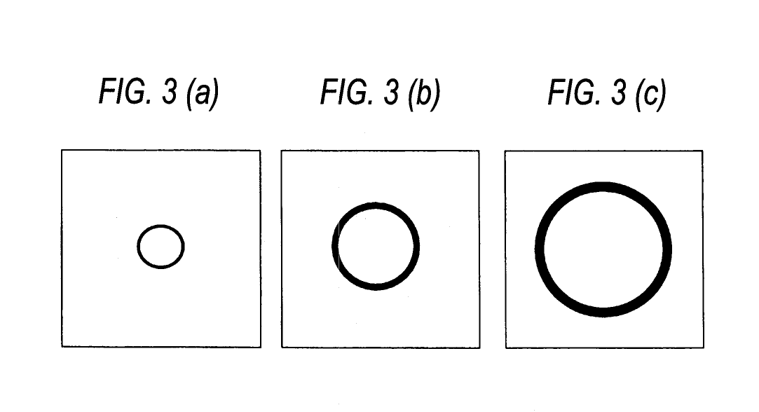

FIG. 3 show examples of training images to be displayed on the display member 1 when the lens 21 is moved. FIG. 3(a) shows a training image to be displayed in the closest position to the trainee 5. FIG. 3(c) shows a training image to be displayed in the farthest position from the trainee 5. FIG. 3(b) shows a training image to be displayed in an intermediate position between those training images. When the training images are formed thus into shapes which have dimensions proportional to their optical distances from the eye position of the trainee 5 and which are similar to one another, there is no substantial change among the dimensions and shapes of the training images observed by the trainee. Thus, the accommodation function of the eye can be controlled easily.

Although the shapes of the training images to be displayed on the plate like display member 1 are formed into shapes similar to one another in the examples of FIG. 3, those shapes may be formed into different shapes. Also in this case, there is no substantial change among the dimensions of the training images occupying the field of view of the trainee. Thus, the accommodation function of the eye can be controlled easily. In addition, since each displayed image has a variation, the trainee can do training unweariedly.

In the visual training apparatus in FIG. 1, the lens 21 which can move with the aid of the motor 23 and the feed screw 22 is used as the optical system 2 having a variable optical characteristic. However, a varifocal lens may be used as the optical system 2 so as to change its magnification to thereby change the training image and the eye position of the trainee.

Although the present invention has been described in detail and with reference to its specific embodiments, it is obvious for those skilled in the art that various changes or modifications can be made on the invention without departing from its spirit and scope.

This application is based on a Japanese patent application (patent application number 2004-207076) filed on Jul. 14, 2004, whose contents are incorporated herein by reference.

### INDUSTRIAL APPLICABILITY

The visual training apparatus according to the present invention is useful as a visual training apparatus or the like in which a training image having dimensions proportional to the distance from the eye position of a trainee is displayed so that the trainee can make accommodation of the trainee's eye easily when the trainee is training.

1. A visual training apparatus for training an accommodation function of an eye, comprising:
a display member that is fixedly disposed to cross a visual axis of a trainee substantially perpendicularly thereto;
an optical system that is disposed between an eye position of the trainee and the display member;
a display control portion that controls display of a training image on the plate-like display member; and
an optical characteristic changing portion that changes an optical characteristic of the optical system to change an optical distance between the eye position of the trainee and the display member,
wherein the display control portion displays on the display member the training image having a dimension proportional to the optical distance between the eye position of the trainee and the display member.

2. The visual training apparatus according to claim 1, wherein the training images displayed on the display member every changed optical distances between the eye position of the trainee and the display member are similar to one another.

3. The visual training apparatus according to claim 1, wherein the optical system includes a lens; and
wherein the optical characteristic changing portion includes a driving portion which moves a position of the lens.

4. The visual training apparatus according to claim 1, wherein the optical system is a varifocal lens.

5. The visual training apparatus according to claim 1, wherein the plate-like display member is a liquid crystal display panel.

6. The visual training apparatus according to claim 1, wherein the plate-like display member is an organic EL display panel in which organic EL devices are formed on a substrate.
